(19) United States
(12) Patent Application Publication
(10) Pub. No.: US 2005/0234467 A1
(43) Pub. Date: Oct. 20, 2005

(54) Screw guide

(76) Inventor: James Rains, Cordova, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/074,530

(22) Filed: Mar. 8, 2005

Related U.S. Application Data
(60) Provisional application No. 60/551,101, filed on Mar. 8, 2004.

(51) Int. Cl. A61B 17/58

(52) U.S. Cl. 606/96

(57) ABSTRACT

Methods and devices for guiding insertion of a surgical screw into a threaded screw hole of a bone plate or similar structure are disclosed. Certain embodiments allow a user to accurately install a locking or non-locking screw into a threaded screw hole along the central axis of the screw hole and may include a drill sleeve that is inserted into a screw guide for pre-drilling along the same axis as the threaded screw hole.

This application claims the benefit of U.S. Provisional Application Ser. No. 60/551,101, entitled “Locking Screw Guide,” filed Mar. 8, 2004, the entire contents of which are hereby incorporated by reference.

### FIELD OF THE INVENTION

This invention relates generally to methods, systems, and devices for use in orthopedic surgery and, more particularly, to a screw guide for installing a locking or non-locking screw into a threaded hole in a bone plate.

### BACKGROUND OF THE INVENTION

Bone fractures lead to complex tissue injuries involving both the bone and the surrounding soft tissue. Anatomical reduction and stable internal fixation with plates and screws are very successful in treating bone fractures. Good bone healing can also result from relative stability, where the clinical outcome is often dependent on obtaining correct length, axis, and rotation of the fractured bone rather than on precise anatomical reduction and absolute stability. To achieve this, while at the same time minimizing the amount of additional soft tissue trauma, treatment of multi-fragmented metaphyseal and diaphyseal fractures with plates and screws was developed. Accordingly, surgical screws are now widely used in orthopedic surgery, leading to the development of other tools for drilling openings for and guiding the installation of surgical screws.

An existing surgical screw guide is part of a combined surgical drill and screw guide that utilizes a common support cylinder for first guiding the drill guide and then guiding the screw with no auxiliary screw guide insert required. This screw guide includes serrated teeth on a bottom end that allows for the user to engage the bone. Another existing screw guide for non-surgical use provides for centering screws and drill bits in countersunk holes. However, neither of these existing screw guides provides for engagement of the guide into a threaded hole, or includes a slot that a screw can pass through after the screw has been partially inserted into the hole and the user wishes to disengage the screw guide from the screw hole.

Accordingly, there remains a need for improved surgical screw guides, including screw guides that allow a user to engage the screw guide into a threaded hole and to safely and effectively disengage the screw guide from the threaded hole while inserting the surgical screw.

### SUMMARY OF THE INVENTION

Certain exemplary embodiments of the present invention provide methods and devices for guiding insertion of a surgical screw into a threaded screw hole of a bone plate or similar structure, allowing a user to accurately install a locking or non-locking screw into a threaded screw hole along the central axis of the screw hole. Certain embodiments may be used with other fasteners, including pegs or other fasteners with heads and shafts. In certain embodiments, a drill sleeve may be inserted into a screw guide to provide a reliable method of pre-drilling along the same axis as the threaded screw hole.

In one embodiment, an apparatus for surgical use comprises a screw guide with a plurality of slots at a proximal end and a distal end threaded to engage a threaded hole of a bone plate and a drill sleeve having a proximal end and a distal end that includes at least one pin that engages one of the plurality of slots at the proximal end of the screw guide when the drill sleeve is inserted within the screw guide. The apparatus may include a drill sleeve with an internal opening that receives an insertion tool to drive the drill sleeve and screw guide.

The screw guide may also include an opening on an outer surface of the screw guide extending along a portion of the longitudinal axis of the screw guide and dimensioned such that a head of a surgical screw can pass through the opening. A C-ring spring, an o-ring, a spring energized O-ring, or ball plungers may be used to retain a drill sleeve within the screw guide. The surgical apparatus may further comprise a drill sleeve that includes an internal opening that receives an end of a hex driver. The screw guide may include a distal end that is tapered. The screw guide may include a plurality of slots are that are u-shaped and spaced apart around the circumference of the proximal end of the screw guide.

In some embodiments, the screw guide includes a slot in an outer surface of the screw guide that extends along a portion of the longitudinal axis of the screw guide and is dimensioned such that a head of a surgical screw can pass through the slot. A distal end of the screw guide may be threaded to engage a threaded hole of a bone plate. The screw guide may also include a plurality of slots that are spaced apart around the circumference of a proximal end of the screw guide. A surgical device may include a screw guide and a drill sleeve having a proximal end and a distal end that includes at least one pin that engages one of a plurality of slots at the proximal end of the screw guide when the drill sleeve is inserted within the screw guide.

In another embodiment of this invention, a method for guiding insertion of a screw into a screw hole of a bone plate comprises inserting a screw guide into the screw hole; installing a screw through the screw guide and screw hole; disengaging the screw guide from the screw hole and passing the screw guide over a head of the screw through a slot in the screw guide; and completing installation of the screw into the bone. Inserting a screw guide into the screw hole may further comprise engaging threads of a distal end of the screw guide with threads of a threaded screw hole. An embodiment may also include coupling a drill sleeve to the screw guide, and may further include creating a pre-drill hole along the axis of the screw hole and removing the drill sleeve from the screw guide prior to installing the screw through the screw guide.

### BRIEF DESCRIPTION OF THE DRAWINGS

FIG. 1 is a perspective exploded view of an embodiment of a screw guide and drill sleeve according to the present invention.

FIG. 2 is a perspective view of an assembled screw guide and drill sleeve and an insertion tool.

FIG. 3 is a side view of the drill sleeve of FIG. 1.

FIG. 4 is a cross-sectional view of the drill sleeve as viewed along cross-section lines 4-4 as shown in FIG. 3.

FIG. 5 is a side view of the screw guide of FIG. 1.

FIG. 6 is a cross-sectional view of the screw guide as viewed along cross-section lines 6-6 as shown in FIG. 5.

FIG. 7 is another side view of the screw guide of FIG. 5, with identification of the screw guide slot.

FIG. 8 is a top view of the screw guide of FIG. 5.

### DETAILED DESCRIPTION OF THE INVENTION

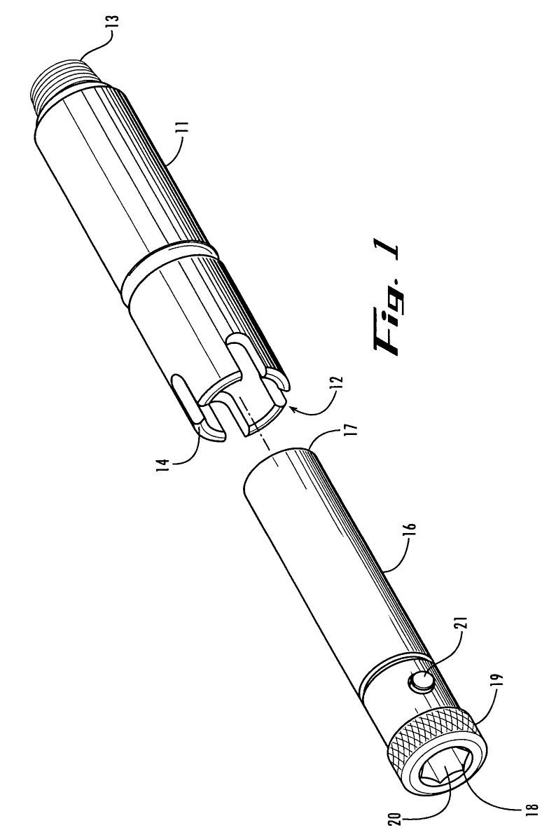

An embodiment of a screw guide 11 and a drill sleeve 16 according to the present invention is shown in FIG. 1. As shown in FIGS. 1, 2, and 5-8, the screw guide 11 has a generally cylindrical configuration. The screw guide 11 has a proximal end 12 and threaded distal end 13 for engaging a threaded screw hole, such as a locking screw hole or other threaded hole on a bone plate. Certain embodiments may be used with other fasteners, including pegs or other fasteners with heads and shafts. The proximal end 12 of the screw guide 11 contains a series of evenly spaced u-shaped slots 14 around the circumference of the proximal end 12 for receipt of a drill sleeve or other tool, such as drill sleeve 16. The screw guide 11, with inner diameter 26 and outer diameter 27, accommodates the insertion of a locking or non-locking screw so that the screw may be inserted within or engaged with a threaded screw hole. Additionally, the screw guide 11 includes a slot 23 that allows for removal of the screw guide 11 over the head of the screw after initial insertion of the screw into the threaded hole.

In the embodiment shown, the threaded distal end 13 of the screw guide 11 is tapered for engagement with a threaded hole, such as the threaded holes commonly used in bone plates. In an alternative embodiment, the distal end of the screw guide can be threaded (not tapered) to engage into a threaded hole (not tapered). In another embodiment, the distal end of the screw guide may be a non-threaded end (tapered or not) configured to mate with a threaded hole. In yet another embodiment, the distal end of the screw guide can be non-threaded (tapered or not) and inserted into a non-threaded hole. In yet another embodiment, the distal end of the screw guide can include a collar that mates with a counterbore on a bone plate at or near the screw hole.

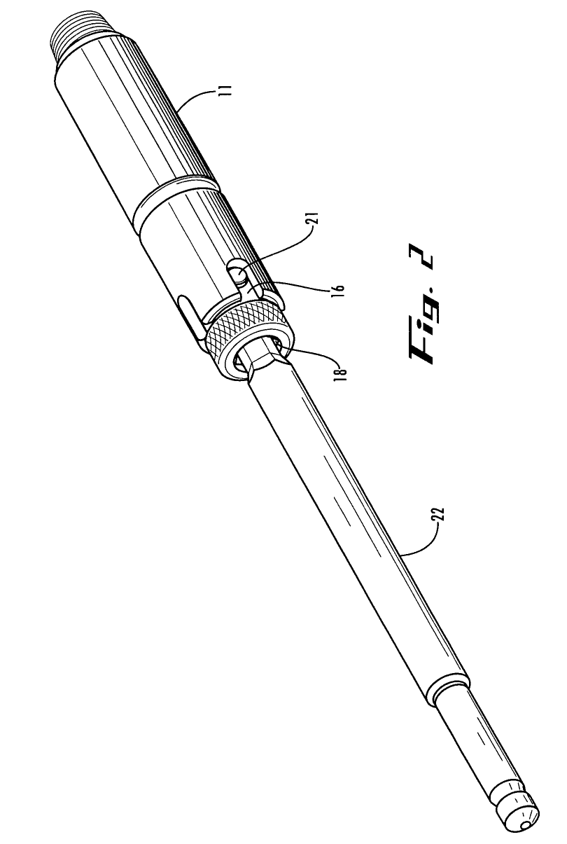

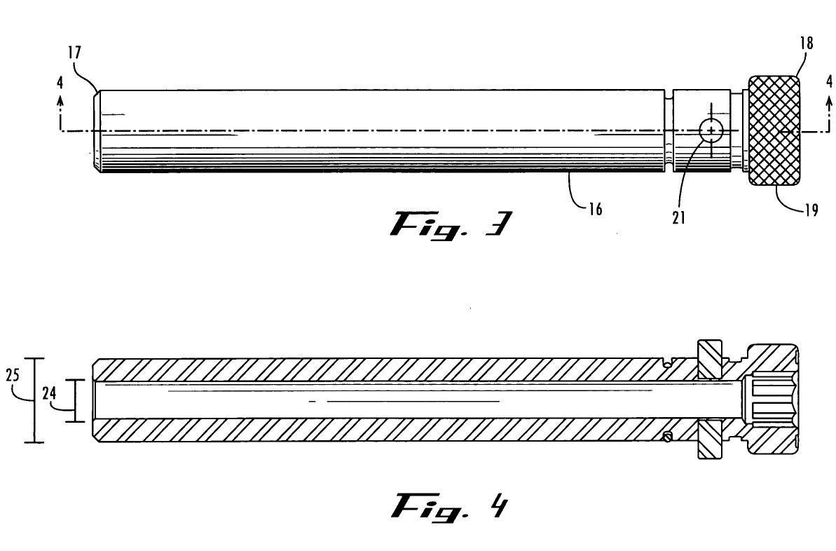

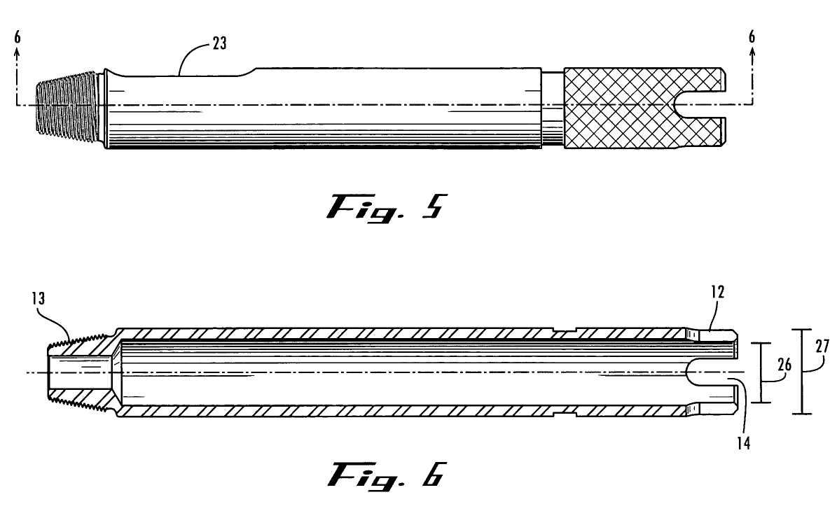

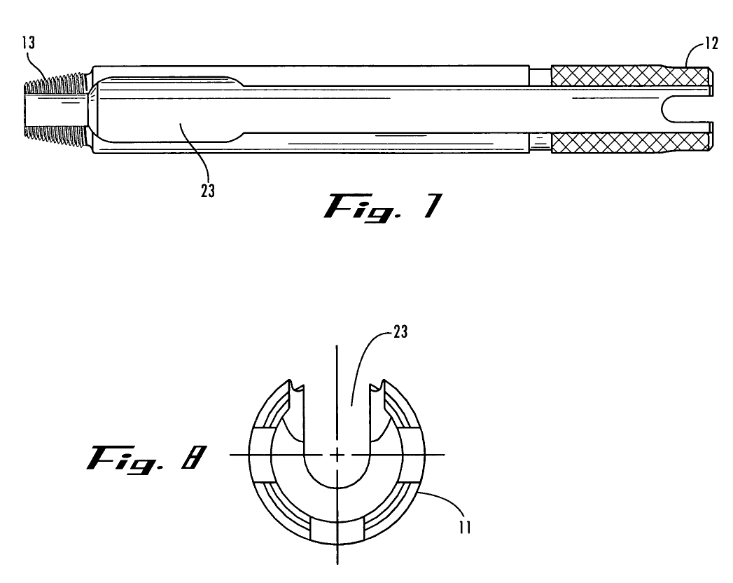

The drill sleeve 16, as shown in FIGS. 1-4, includes a distal end 17 that may be inserted into the screw guide 11. The proximal end 18 of the drill sleeve 16 includes a knurled cap 19 with an internal hexagonal opening 20 for receipt of an insertion tool, such as insertion tool 22 shown in FIG. 2 and described further below. The drill sleeve 16 also includes a raised pin 21 to engage one of the slots 14 on the proximal end 12 of the screw guide to assist in turning the screw guide 11. The drill sleeve 16 is generally cylindrical with an inner diameter 24 and outer diameter 25 along the length of the drill sleeve 16 with openings on the distal end 17 and the proximal end 18. The drill sleeve 16 is inserted longitudinally into screw guide 11 along the central axis of the screw guide 11 and drill sleeve 16. In one embodiment, the drill sleeve 16 is retained in the screw guide 11 by a C-ring spring that provides friction between the screw guide 11 and the drill sleeve 16. In other embodiments, a rubber O-ring, spring energized O-ring, ball plunger or other mechanisms may be used to retain drill sleeve 16 within screw guide 11.

Referring to FIG. 2, the assembly of the screw guide 11 and the drill sleeve 16 can be accomplished by hand or with the insertion tool 22. In one embodiment, insertion tool 22 is a hex driver, but numerous other devices may be used as an alternative to a hex driver, provided that such devices are capable of engaging the drill sleeve and driving the drill sleeve/screw guide assembly, which is well understood by those skilled in the art. In the embodiment shown in FIG. 2, the insertion tool 22 engages the proximal end 18 of the drill sleeve 16 and the pin 21 of the of the drill sleeve 16 engages one of the slots 14 on the screw guide 11 which allows the insertion tool 22 to drive the screw guide 11 for insertion into a threaded hole (not shown).

In an embodiment of a method according to the present invention, once the drill sleeve and screw guide are assembled and the assembly is threaded into a threaded screw hole (such as the threaded screw holes commonly found in bone plates), a pre-drill hole may be created in a bone to which the bone plate is being secured along the axis of the threaded screw hole. The drill sleeve 16 may then be removed and a locking or non-locking screw may be inserted through screw guide 11 and into the threaded screw hole. The screw may continue to be installed until the operator desires to disengage the screw guide from the hole, which generally is desirable just prior to full insertion of the screw. For example, in one embodiment, the screw is installed until the indicator mark on the screw drive shaft is parallel with the top of screw guide 11. This or a similar feature informs the user that the screw is near final insertion and that the screw guide 111 needs to be removed so that the head of the screw can be fully seated in the threaded screw hole. The screw guide 11 is then disengaged from the threaded screw hole, and is removed by passing it over the screw via the opening slot 23 (as best shown in FIGS. 5 and 7) in the distal end 13 of the screw guide 11. The opening slot 23 is also dimensioned to allow for the screw guide to be passed over an insertion tool, such as a hex driver. After the removal of the screw guide 11, the screw is fully inserted into the threaded hole and the bone. For self-drilling screws, the pre-drill step is not necessary.

The foregoing description of certain exemplary embodiments of the invention has been presented only for the purposes of illustration and description and is not intended to be exhaustive or to limit the invention to the precise forms disclosed. Many modifications and variations are possible in light of the above teaching. For example, when using cannulated screws, a K-wire sleeve is used with screw guide 11 instead of drill sleeve 16. This provides a stable mechanism for inserting a K-wire along the central axis of a threaded screw hole. As another example, alternatives well known to those skilled in the art may be used in lieu of the pin of drill sleeve 16 and slots of screw guide 11 to allow for the drill sleeve to drive the screw guide once the insertion tool is used to turn the sleeve. Yet another example is that the distal end of screw guide 11 may be tapered but not threaded, giving approximate alignment with the threaded screw hole, but not as exact as using a threaded distal end.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and certain exemplary embodiments described therein.

1. An apparatus for surgical use comprising:
a screw guide with a plurality of slots at a proximal end and a distal end threaded to engage a threaded hole of a bone plate; and
a drill sleeve having a proximal end and a distal end that includes at least one pin that engages one of the plurality of slots at the proximal end of the screw guide when the drill sleeve is inserted within the screw guide.

2. The apparatus of claim 1, wherein the drill sleeve further comprises an internal opening that receives an insertion tool to drive the drill sleeve and screw guide.

3. The apparatus of claim 1, wherein the screw guide further comprises an opening on an outer surface of the screw guide extending along a portion of the longitudinal axis of the screw guide and dimensioned such that a head of a surgical screw can pass through the opening.

4. The apparatus of claim 1, further comprising a C-ring spring that retains the drill sleeve within the screw guide.

5. The apparatus of claim 1, further comprising an o-ring that retains the drill sleeve within the screw guide.

6. The apparatus of claim 1, further comprising ball plungers that retain the drill sleeve within the screw guide.

7. The apparatus of claim 1, further comprising a hex driver and wherein the drill sleeve further comprises an internal opening that receives an end of the hex driver.

8. The apparatus of claim 1, wherein the distal end of the screw guide is tapered.

9. The apparatus of claim 1, wherein the plurality of slots are u-shaped and spaced apart around the circumference of the proximal end of the screw guide.

10. A method for guiding insertion of a screw into a screw hole of a bone plate, the method comprising:
inserting a screw guide into the screw hole;
installing a screw through the screw guide and screw hole;
disengaging the screw guide from the t screw hole and passing the screw guide over a head of the screw through a slot in the screw guide; and
completing installation of the screw into the bone.

11. The method of claim 10, wherein inserting the screw guide into the screw hole further comprises engaging threads of a distal end of the screw guide with threads of the screw hole.

12. The method of claim 10, further comprising coupling a drill sleeve to the screw guide.

13. An apparatus for surgical use comprising:
a screw guide having a slot in an outer surface of the screw guide that extends along a portion of the longitudinal axis of the screw guide and is dimensioned such that a head of a surgical screw can pass through the slot.

14. The apparatus of claim 13, wherein the screw guide further comprises a distal end threaded to engage a threaded hole of a bone plate.

15. The apparatus of claim 14, wherein the screw guide further comprises a plurality of slots spaced apart around the circumference of a proximal end of the screw guide.

16. The apparatus of claim 15, further comprising a drill sleeve having a proximal end and a distal end that includes at least one pin that engages one of the plurality of slots at the proximal end of the screw guide when the drill sleeve is inserted within the screw guide.

17. The apparatus of claim 16, wherein the drill sleeve further comprises an internal opening that receives an insertion tool to drive the drill sleeve and screw guide.

18. The apparatus of claim 16, further comprising a C-ring spring, o-ring, or ball plungers that retains the drill sleeve within the screw guide.

19. The apparatus of claim 13, wherein the distal end is tapered to engage a hole in a bone plate.

20. The apparatus of claim 13, wherein the distal end of the screw guide is tapered and unthreaded.
